(19) United States
(12) Patent Application Publication
(10) Pub. No.: US 2007/0128272 A1
(43) Pub. Date: Jun. 7, 2007

(54) Multi-vitamin and mineral supplement

(76) Inventors: Horst G. Zerbe, Hudson (CA); Nadine Paiement, Montreal (CA); Pompilia Ispas-Szabo, Greenfield Park (CA)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/635,361

(22) Filed: Dec. 7, 2006

Related U.S. Application Data
(60) Provisional application No. 60/748,298, filed on Dec. 7, 2005.

(51) Int. Cl. A61K 9/48; A61K 9/20; A61K 9/14

(52) U.S. Cl. 424/451; 424/464; 424/484

(57) ABSTRACT

A nutritional solid oral dosage form for administering a calcium nutrient with an iron nutrient includes enterically coated particles, granules or pellets containing a calcium nutrient. An iron nutrient is incorporated into an immediate release matrix. The coated calcium nutrient may be located in a core surrounded by the immediate release matrix or distributed in the immediate release matrix. The coating and matrix are formulated to allow immediate release of the iron nutrient in an upper part of the gastrointestinal tract and delayed release of the calcium nutrient until the remainder of the dosage forms reaches a higher pH region of the gastrointestinal tract.

### CROSS-REFERENCE TO RELATED APPLICATION

This application claims priority under 35 U.S.C. § 119(e) on U.S. Provisional Application No. 60/748,298 entitled MULTI-VITAMIN AND MINERAL SUPPLEMENT, filed Dec. 7, 2005, the entire disclosure of which is incorporated herein by reference.

### FIELD OF THE INVENTION

This invention relates to dietary supplements, and more particularly to nutritional supplements that exhibit enhanced iron and calcium bioavailability.

### BACKGROUND OF THE INVENTION

It is well recognized that concurrent calcium and iron supplementation using a single oral dosage form has been difficult due to undesirable interactions between these minerals, which reduce bioavailability. The depression of iron absorption by high levels of calcium was recognized as early as 1940. Since then, various groups have repeatedly confirmed the significant inhibition of iron absorption by calcium. Adverse effects of calcium, including a decrease in hemoglobin regeneration, reduced whole blood iron retention and delayed restoration of tissue and blood iron levels have been reported. For many decades, orange juice has been recognized as an enhancer to iron absorption. Specifically, there are reports in the literature that orange juice increases iron bioavailability. In addition, various forms of calcium and iron and have been suggested to improve bioavailability. In the case of calcium, these include lactate, gluconate, carbonate, citrate and citrate malate salts. Suggested iron sources have included iron sucrate, and various ferrous and/or ferric coordination complexes of ammonium salts, citrates, tartrates, amines, sugar and glycerin.

U.S. Pat. No. 4,994,283 discloses iron-calcium mineral supplements that are alleged to exhibit enhanced bioavailability. The ability to achieve enhanced conjoint bioavailability of iron and calcium is allegedly achieved by coadministering a source of calcium and a source of iron with a potentiating amount of citrate or tartrate, or mixtures thereof, and ascorbate. More specifically, it is alleged that citric acid (or citrates) and tartartic acid (or tartrates) partially alleviate the inhibitory effect of calcium on iron, and mixtures of citric/ascorbic acid (or citrate/ascorbate mixtures), or tartaric acid/ascorbic acid (or tartrates/ascorbate) or mixtures, overcome the inhibitory effect.

It has been previously proposed to provide a multi-vitamin and mineral dietary supplement containing a divalent mineral (e.g., calcium or magnesium) and iron, wherein interactions between the minerals are obviated by a controlled release dosage form. More specifically, U.S. Pat. No. 4,752,479 discloses a unit dose formulation including an outer layer containing a divalent mineral component and an inner core containing a bioavailable iron component in a controlled release form. It is reported that by virtue of its release into the upper gastrointestinal tract, the calcium and/or magnesium component can be substantially absorbed into the body of the host prior to the controlled continuous release of the bioavailable iron component lower in the gastrointestinal tract. It is claimed that the interfering effect of calcium and magnesium upon the absorption of iron is minimized using the controlled release formulation.

U.S. Pat. No. 4,431,634 discloses a method that allegedly enhances absorption of iron in a multi-mineral, iron-supplement preparation by limiting the quantities of oxides and carbonates of calcium and magnesium administered in these preparations to not more than 300 milligrams and 75 milligrams, respectively, per unit dosage based upon the weight of elemental calcium and magnesium in the oxide and carbonate salts.

A multi-vitamin and mineral supplement for pregnant women is disclosed in U.S. Pat. No. 6,228,338. The disclosed supplement is said to be tailored to simultaneously meet nutritional requirements of both a developing fetus and mother during each of the individual trimesters of pregnancy, wherein both an iron source and a calcium source are present in the supplement for the third trimester. The iron source in the supplement, along with vitamin C and folic acid components are preferably coated with a suitable controlled release film-forming material.

### SUMMARY OF THE INVENTION

The present invention provides an innovative solution to the problem of preventing and/or reducing interactions between a calcium nutrient and an iron nutrient present in a single dosage form by isolating the calcium nutrient in an enterically coated particle, granule or pellet. The use of an enterically coated particle, granule or pellet containing a calcium nutrient allows an iron nutrient to be formulated in an immediate release portion of a dosage form, while release of the calcium nutrient is delayed until the enterically coated particle, granule or pellet reaches the higher pH region (above 5.5 pH) of the gastrointestinal tract (i.e., the upper part of the intestine). This controlled or delayed release of the calcium nutrient prevents or reduces simultaneous presence of the iron calcium nutrient, thereby reducing or eliminating interactions between the two nutrients that would otherwise reduce bioavailability and absorption.

These and other features, advantages and objects of the present invention will be further understood and appreciated by those skilled in the art by reference to the following specification and claims.

### DESCRIPTION OF PREFERRED EMBODIMENTS

Compositions and nutritional supplements in accordance with this invention include enterically coated particles, granules or pellets that contain a calcium nutrient that is retained in the coated particles, granules or pellets after oral administration, until the particles, granules or pellets reach the intestine, whereupon the calcium nutrient is released. An enterically coated particle, granule or pellet is a particle, granule or pellet that has been coated with a substantially continuous or encapsulating enteric coating. An enteric coating is a polymer film coating that prevents the particle, granule or pellet from dissolution at low gastric pH levels, and dissolves and/or disintegrates at the higher pH (e.g., about 5.5) in the upper part of the intestine, thereby preventing release of the calcium nutrient in the stomach and/or other parts of the upper gastrointestinal tract, and allowing release of the divalent mineral nutrient in the upper portion of the intestine. Enteric coatings are well-known and commonly utilized to protect and prevent release of pharmaceutically active compounds until they reach the duodenum. Examples of suitable enteric coatings include those compositions containing a pH-sensitive methacrylate copolymer (e.g., Eudragit® L 30D, Eudragit® S 100, Kollicoat® MAE and Kollicoat® EMM 30 commercially available enteric coating compositions).

The pellets used in the compositions and nutritional supplements of the invention are solid formulations, including, but not limited to, tablets, bricks, briquettes, bars, balls or blocks. In general, the pellets are characterized as comprising a coherent mass of shaped material including the calcium nutrient. Preparation of pellets is well-known in various related arts (e.g., pharmaceuticals and detergents), and typically involves incorporating one or more binders within the body of the pellets by employing any of various suitable methods. Conventional pellet extruders or spheronizers (marumerizers) may be employed for making pellets. Examples of other suitable methods for preparing pellets for use in the invention include forming an aqueous slurry of the pellet materials including one or more binders, spray drying the slurry to give a granular product and then compacting these granules in a pelletizing machine to form pellets. Alternatively, pellets suitable for use in the invention may be prepared by grinding together a dry mixture of the pellet materials, including one or more solid binders, and then compacting this mixture in a pelletizing machine to form pellets. As another alternative, suitable pellets for use in the invention may be prepared by spraying one or more binders into the other pellet materials in powder form and then compacting the combined materials to form pellets.

Granules include substantially unshaped or randomly shaped agglomerations of particles, and may, for example, by prepared using known fluid bed granulation techniques.

The term “particle” is used herein to encompass any enterically coatable material that can be dispersed in an oral dosage form matrix material, and which cannot be fairly described as a pellet or granule.

The particles, granules and/or pellets containing a calcium nutrient typically have a size, based on the major or largest dimension of the particle, granule or pellet in the range of 20-60 mesh, preferably 200-800 microns, after coating.

Desirably, the particles, granules or pellets include, in addition to the calcium nutrient, a nutritionally acceptable acid that provides a microenvironment which facilitates conversion of the calcium nutrient into a form exhibiting enhanced bioavailability. Nutritionally acceptable acids include, but are not limited to, various well-known food-grade acids, such as citric acid, tartaric acid, malic acid, succinic acid, ascorbic acid, fumaric acid, phosphoric acid, gluconic acid, acetic acid, tannic acid, lactic acid and glycolic acid.

The calcium nutrient is typically a salt. Suitable salt forms include lactates, gluconates, citrates and carbonates.

Iron nutrients that may be employed include ferrous or ferric ion sources, or a combination of both, with ferrous ion sources being preferred. Examples of iron sources include ferrous fumarate, ferrous gluconate, ferrous sulfate and iron-polysaccharide.

The enterically coated particles, granules or pellets containing a calcium nutrient may be incorporated into various oral dosage forms, along with other nutrients (e.g., vitamins and minerals), excipients and adjuvants, such as capsules or tablets. A preferred oral dosage form, described in greater detail herein, is a core/mantle tablet arrangement in which a portion of the enterically coated pellets, and optionally other nutrients, are incorporated into an extended release core, and another portion of the enterically coated pellets is incorporated, along with an iron source in an immediate release mantle or layer that surrounds the core. The iron source, and other nutrients in the mantle that are external to the enterically coated particles, granules or pellets are immediately released in the stomach, whereas the enterically coated particles, granules or pellets of the mantle delay release of the calcium nutrient until the particles, granules or pellets reach the duodenum. Meanwhile, the core, which may comprise a combination of pH dependent (e.g., enteric) polymeric binders and pH independent polymeric binders can slowly release the remaining enterically coated particles, granules or pellets and other nutrients in the core over an extended period of time.

According to this invention, particles, granules or pellets containing a calcium nutrient (e.g., calcium carbonate) and an intestinal absorption enhancer (e.g., a food-grade acid) are coated with a gastric resistant membrane (e.g., an enteric coating). The enterically coated pellets can be divided in two groups, each group comprising different vitamins and minerals together with different pharmaceutical adjuvants. One group may consist of a calcium source, water-insoluble vitamins (e.g., vitamin D and vitamin E), iodine, zinc oxide and a disintegrant (composition included in Table I under the name of CORE). This mixture may be compressed to form a solid core, which can be transferred and well centered into a die. The second group of enterically coated pellets can be mixed with vitamin A, riboflavin (vitamin B₂), thiamin mononitrate (vitamin B ₁), cyanocobalamin (vitamin B ₁₂), ascorbic acid (vitamin C), vitamin K, niacin and iron (ferrous form). Pharmaceutically acceptable excipients (i.e. disintegrants, lubricants, etc.) may also be incorporated in this second mixture that can be transferred into conventional equipment and compressed around the core.

As another alternative, tablets may be prepared by dispersing the enterically coated particles, granules or pellets containing a calcium nutrient in an immediate release matrix containing an iron nutrient. The matrix may contain vitamins and/or other minerals. Similarly, the enterically coated particles, granules or pellets may contain vitamins and/or other minerals.

As yet another alternative, tablets may be prepared having a core matrix containing at least one vitamin nutrient, and a mantle layer encompassing the core and including a mantle matrix into which are dispersed enterically coated particles, granules or pellets containing a calcium nutrient. Also contained in the mantle matrix is an iron nutrient available for immediate release. Any of the core, mantle matrix and/or the enterically coated particles, granules or pellets may contain additional nutrients, e.g., vitamins and/or minerals. Water-soluble nutrients that do not interfere with the bioavailability of iron are preferably located in an immediate release mantle matrix. Other vitamins and/or minerals that interfere with the bioavailability of iron are preferably located in the coated pellets, granules or particles or in the core, which may include a combination of polymeric binders that facilitate controlled release.

The finished tablets may be film-coated using a conventional film-coating technique for improving appearance and acceptability.

The present invention is distinguishable from other multivitamin and mineral dosage forms by size and shape characteristics of the tablet in association with its therapeutic properties. The invention is capable of incorporating various nutrients (i.e., vitamins and minerals) required for prenatal therapy into an easy to swallow tablet that becomes smaller in the stomach (about two-thirds of the total tablet mass can be represented by the mantle which disintegrates rapidly leaving behind a smaller unit—the tablet core).

In one embodiment of the invention, the enterically coated particles, granules or pellets are prepared using conventional coating equipment (bottom spray) to achieve delayed release and create a physical and temporal separation between calcium and iron with a beneficial effect on their absorption and bioavailability.

Various pharmaceutical excipients may be used in the process, including pH dependent polymers, pH independent polymers, permeability enhancers, bulking agents, anti-foam agents, plasticizers, anti-tacking agents and other adjuvants. Purified water may be used as a solvent to provide a process in which all steps are free of organic solvents.

The dry powders of calcium carbonate and citric acid are transferred in a fluidized bed granulator (Aeromatic Strea type 1) equipped with a bottom spray unit. The powders are warmed between 30-40° C. for 5 minutes followed by spraying of a polymeric suspension. Typical processing/temperature conditions for an Aeromatic-Fielder Strea-1™ fluid bed drier are as follows: inlet air temperature 30-50° C., outlet air temperature 25-40° C., flow rate 4 g/min, process time 60-90 minutes. The pellets are dried after at higher temperature between 45-55° C. for other 5-15 minutes.

Various processing conditions (i.e. temperature gradients, flow rate, weight gain etc.) may be adjusted as desired to control the gastric resistance of the coated pellets (Example 4).

A curing step at 22° C. for 24 hours or at 40° C. for 2 hours and an extra 2 hours at room temperature was allowed for pellet relaxation.

The enteric coating typically represents between 15% to 30% of the total mass of the enterically coated pellets.

In another embodiment of the invention, the enterically coated pellets are dry blended with selected vitamins and supplements together with a disintegrant and compression adjuvants (e.g., lubricant). These preparations can be compressed to obtain the tablet core. Various ratios of pellet weight to the weight of the external phase or core matrix (core material that is not contained in the pellets) may be employed, with a preferred ratio being illustrated in Example 5. Preferred compression parameters were between 7-15 kN for a core hardness not exceeding 10 Kpounds.

A second part of the enterically coated pellets can be incorporated in a composition used to form a mantle around the core. A dry mixing procedure is used to prepare a composition comprising the second part of the enterically coated pellets, and ingredients external to the pellets which form a mantle matrix in which the pellets are distributed. The external or mantle matrix ingredients may include vitamin B₁, vitamin B₂, vitamin B₆, vitamin B₁₂, niacin, folic acid, a source of ferrous and/or ferric ions, and pharmaceutically acceptable excipients (e.g., binders, disintegrants, lubricants, flowability agents, etc.) The total mass of the mantle may vary between 400-1000 mg (Example 5).

In another embodiment of the invention the precompressed cores may be transferred into a dry coater apparatus and well centered into a die. The mantle preparation is fed into the die and a second compression step may be employed to ensure the production of a final tablet with a hardness between 5 to 15 Kpounds.

The tablets are film-coated for protection against moisture and light by using well-known polymeric barriers combined with pigments, plasticizers and anti-tacking agents. Those skilled in the art will select such systems from commercially available products based on cellulose derivatives (e.g., Opadry® white or clear, Opadry® II and Opadry® AMB).

Tablets containing water-insoluble vitamins and a portion of the enterically coated pellets may be incorporated in a layer (tablet core) that is not protected by a functional coating. This core is surrounded by a second layer or mantle, incorporating water-soluble vitamins, niacin, folic acid, the remaining enterically coated pellets and selected pharmaceutical excipients.

The pellets containing a divalent mineral nutrient (e.g., calcium) are prepared by association of calcium carbonate and desirably a nutritionally acceptable acid using polymeric binders. The polymeric binders may comprise a combination of pH dependent and pH independent polymers. The role of the nutritionally acceptable acid is to increase the absorption of the divalent nutrient (e.g., calcium) at a specific site in the intestine by ensuring an acidic microenvironment, that will facilitate conversion of the divalent mineral nutrient to a more absorbable form.

The invention is illustrated by the following non-limitative Examples:

### EXAMPLE 1

| | Composition |
|---|---|
| Ingredients | (%) |
| Calcium carbonate | 70.0 |
| Citric acid | 7.0 |
| Eudragit ® NE | 10.0 |
| Eudragit ® L 30D | 10.0 |
| Excipients | 3.0 |

A mixture of calcium carbonate and citric acid is sprayed with an aqueous dispersion of the methacrylate copolymers A and B (Eudragit® NE and Eudragit® L 30D) in a fluid bed and then dried. The coated pellets are cured at 40° C. for 2 hours and allowed to set for an additional 2 hours at room temperature for relaxation.

### EXAMPLE 2

| | Composition |
|---|---|
| Ingredients | (%) |
| Calcium carbonate | 70.0 |
| Citric acid | 7.0 |
| Eudragit ® NE 30D | 5.0 |
| Eudragit ® S 100 | 15.0 |
| Excipients | 3.0 |

A mixture of calcium carbonate and citric acid is sprayed with an aqueous dispersion of the methacrylate copolymers A and B (Eudragit® NE 30D and Eudragit® S 100) in a fluid bed and then dried. The coated pellets are cured at 40° C. for 2 hours and allowed to set or an additional 2 hours at room temperature for relaxation.

### EXAMPLE 3

| | Composition |
|---|---|
| Ingredients | (%) |
| Calcium carbonate | 70.0 |
| Citric acid | 7.0 |
| Kollicoat MAE | 15.0 |
| Kollicoat EMM 30 | 5.0 |
| Excipients | 3.0 |

A mixture of calcium carbonate and citric acid is sprayed with an aqueous dispersion of poly ethylacrylate, methyl methacrylate in a fluid bed and then dried. The coated pellets are cured at 50° C. for 2 hours and allowed to set for an additional 2 hours at room temperature for relaxation.

Selected coating conditions were employed to generate a gastric-resistant core from coated pellets (Example 4).

| Process parameters | Trial 1 | Trial 2 | Trial 3 |
|---|---|---|---|
| Coating temperature (C.) | 40 | 40 | 45 |
| Nozzle size (mm) | 0.8 | 0.8 | 1.1 |
| Atomized pressure (bar) | 0.6 | 0.8 | 0.8 |
| Flow rate (g/min) | 5 | 4 | 5 |
| Weight gain (%) | 20 | 20 | 25 |

The Ca pellets were used for both the core preparation and the mantle preparation. Partitioning of these pellets, between the core and mantle, was done at different ratios as illustrated in Example 5.

### EXAMPLE 5

| | Core/Mantle |
|---|---|
| Ca gastric resistant pellets | 1:1 |
| Ca gastric resistant pellets | 1:2 |
| Ca gastric resistant pellets | 1:3 |
| Ca gastric resistant pellets | 1:4 |

A core-mantle tablet containing enterically coated calcium nutrient granules dispersed in both a core matrix and a mantle matrix is prepared.

The granules containing the calcium nutrients are prepare by employing a wet granulation technique, followed by drying, and enteric coating of the dried granules. The coating has the following formula.

ENTERIC COATING COMPOSITION

| Ingredient | w/w % |
|---|---|
| Eudragit ® NE30D | 6.00 |
| Eudragit ® L30D55 | 9.00 |
| Glycerol monostearate | 0.58 |
| Triethyl Citrate | 1.11 |
| Polysorbate (Tween 80) | 0.23 |
| Water | 83.08 |
| Total | 100 |

The granules may be coated using a fluid bed coating apparatus. The enterically coated granules have the following formula.

ENTERICALLY COATED CALCIUM GRANULES

| Ingredient | mg/Tablet | % |
|---|---|---|
| Calcium Carbonate | 556.906 | 70.00 |
| Citric Acid | 55.691 | 7.00 |
| Eudragit ® NE30D | 63.64 | 8.00 |
| Eudragit ® L30D55 | 95.46 | 12.00 |
| Glycerol Monostearate | 7.21 | 0.91 |
| Triethyl Citrate | 13.8 | 1.73 |
| Polysorbate (Tween) 80 | 2.86 | 0.36 |
| Total | 795.567 | 100.0 |

The core is prepared by compressing the following formulation.

CORE COMPOSITION

| Ingredient | mg/Tablet |
|---|---|
| Vitamin D | 6.000 |
| Vitamin E | 63.953 |
| Coated Calcium Granules | 198.892 |
| Iodine | 3.750 |
| Zinc | 16.517 |
| HPMC acetate succ. | 10.000 |
| Mg stearate | 3.000 |
| Total | 302.112 |

Compressed over the core is a mantle layer having the following formula.

MANTLE COMPOSITION

| Ingredient | mg/Tablet |
|---|---|
| Vitamin C | 129.897 |
| Vitamin B₁ | 3.582 |
| Vitamin B₂ | 3.481 |
| Vitamin B₆ | 3.307 |
| Vitamin B₁₂ | 9.200 |
| Niacin | 20.420 |
| Folic Acid | 12.000 |
| Iron | 29.768 |
| Coated Calcium Granules | 596.675 |
| Ac-di-sol | 30.000 |
| Mg Stearte | 8.000 |
| Total | 846.330 |

A final aesthetic/functional coating layer is applied over the mantle layer. The final coating composition has the following formula.

FINAL COATING COMPOSITION

| Ingredient | w/w % |
|---|---|
| Water | 90 |
| Clear Opadry | 9 |
| Sucrose | 0.9 |
| Polyethylene Glycol | 0.09 |
| Total | 100 |

By dispersing a portion of the enterically coated calcium granules in the core and another portion in the matrix, it is possible to release a delayed burst or pulse of calcium nutrient at an upper part of the intestine, and provide a sustained release of calcium nutrient throughout a lower portion of the intestine. However, depending on the desired release profile, all or substantially all of the enterically coated calcium granules may be located in either the core or the mantle. In general, vitamins and other mineral nutrients that are not water-soluble or that could interfere with iron absorption are located in the core, or incorporated into the enterically coated granules or pellets, provided they do not also interfere with calcium absorption, and vitamin and mineral nutrients that are water-soluble or that could interfere with calcium absorption are incorporated into the mantle along with the iron.

The above description is considered that of the preferred embodiments only. Modifications of the invention will occur to those skilled in the art and to those who make or use the invention. Therefore, it is understood that the embodiments described above are merely for illustrative purposes and not intended to limit the scope of the invention, which is defined by the following claims as interpreted according to the principles of patent law, including the doctrine of equivalents.

The invention claimed is:

1. A nutritional solid oral dosage form, comprising:
a matrix material in which are dispersed enterically coated particles, granules or pellets containing a calcium nutrient.

2. The dosage form of claim 1, wherein the enterically coated particles, granules or pellets further comprise a nutritionally acceptable acid.

3. The dosage form of claim 1, wherein the nutritionally acceptable acid is citric acid.

4. The dosage form of claim 1, wherein the nutritionally acceptable acid is selected from the group consisting of tartaric acid, malic acid, succinic acid, ascorbic acid, fumaric acid, phosphoric acid, gluconic acid, acetic acid, tannic acid, lactic acid, and glycolic acid.

5. The dosage form of claim 1, further comprising an iron nutrient.

6. The dosage form of claim 1, wherein the calcium nutrient is a calcium salt.

7. The dosage form of claim 1, which is in the form of a tablet.

8. The dosage form of claim 1, which is in the form of a capsule.

9. The dosage form of claim 1, further comprising vitamin D, folic acid, vitamin B₁₂, vitamin B₆ and vitamin B₁.

10. The dosage form of claim 1, wherein the calcium nutrient is selected from the group consisting of calcium lactate, calcium gluconate and calcium citrate.

11. The dosage form of claim 7, further comprising at least one disintegrant.

12. The dosage form of claim 1, wherein the particles, granules or pellets have a size of 200 micrometers to 800 micrometers.

13. The dosage form of claim 1, wherein the enteric coating comprises from about 15 to about 30% of the enterically coated particles, granules or pellets.

14. A nutritional tablet dosage form, comprising:
a core including a core matrix and a plurality of enterically coated particles, granules or pellets dispersed in the core matrix, the enterically coated pellets in the core containing a calcium nutrient and a nutritionally acceptable acid; and
a mantle layer encompassing the core, the mantle layer including a mantle matrix and a plurality of enterically coated particles, granules or pellets dispersed in the mantle matrix, the enterically coated particles, granules or pellets in the mantle layer containing a calcium nutrient and a nutritionally acceptable acid, the mantle matrix including an iron nutrient.

15. The tablet of claim 14, wherein the calcium nutrient is a calcium salt.

16. The tablet of claim 14, wherein the nutritionally acceptable acid is citric acid.

17. The tablet of claim 14, wherein the nutritionally acceptable acid is selected from the group consisting of tartaric acid, malic acid, succinic acid, ascorbic acid, fumaric acid, phosphoric acid, gluconic acid, acetic acid, tannic acid, lactic acid, and glycolic acid.

18. The tablet of claim 14, further comprising vitamin D, folic acid, vitamin B₁₂, vitamin B₆ and vitamin B ₁.

19. The tablet of claim 14, wherein the calcium nutrient is calcium carbonate.

20. The tablet of claim 14, wherein the calcium nutrient is selected from the group consisting of calcium lactate, calcium gluconate and calcium citrate.

21. The tablet of claim 14, wherein the mantle matrix further comprises a disintegrant.

22. The tablet of claim 14, wherein the particles, granules or pellets have a size of 200 micrometers to 800 micrometers.

23. The tablet of claim 14, wherein the enteric coating comprises from about 15 to about 30% of the enterically coated particles, granules or pellets.

24. The tablet of claim 14, wherein the core further comprises a combination of an enteric polymer binder and a pH independent polymer binder.

25. A nutritional tablet dosage form, comprising:
a core including a core matrix containing at least one vitamin nutrient;
a mantle layer encompassing the core, the mantle layer including a mantle matrix and a plurality of enterically coated particles, granules or pellets dispersed in the mantle matrix, the enterically coated particles, granules or pellets in the mantle layer containing a calcium nutrient and a nutritionally acceptable acid, the mantle matrix including an iron nutrient.

26. The tablet of claim 25, wherein the vitamin nutrient in the core is selected from vitamin D, vitamin A, vitamin E, vitamin K, and combinations of these vitamins.
